(19) United States
(12) Patent Application Publication
(10) Pub. No.: US 2026/0179221 A1
(43) Pub. Date: Jun. 25, 2026

(54) MONITORING METHOD FOR ON-BOARD MEDICAL DEVICE, COMPUTER DEVICE, AND STORAGE MEDIUM

(71) Applicant: Shanghai United Imaging Healthcare Co., Ltd., Shanghai (CN)

(72) Inventor: Ye Kuang, Shanghai (CN)

(21) Appl. No.: 19/417,262

(22) Filed: Dec. 11, 2025

(30) Foreign Application Priority Data
Dec. 20, 2024 (CN) 202411899857.1

(51) Int. Cl. G06T 7/00; A61B 6/00; A61B 6/03; A61B 6/58; G06T 7/33; G06T 7/70; G16H 40/63

(52) U.S. Cl. CPC G06T 7/0012; A61B 6/032; A61B 6/037; A61B 6/5235; A61B 6/582; G06T 7/337; G06T 7/70; G16H 40/63; G06T 2207/10081; G06T 2207/10104; G06T 2207/10108; G06T 2207/20221; G06T 2207/30168; G06T 2207/30232; G06T 2207/30244

(57) ABSTRACT

The present disclosure relates to a monitoring method for an on-board medical device, a computer device, and a storage medium. The method includes: acquiring environmental information of the on-board medical device; in response to the environmental information exceeding a preset environmental threshold, controlling the first imaging assembly to image an imaging object to obtain a first image, and controlling the second imaging assembly to image the imaging object to obtain a second image; performing an accuracy check on the on-board medical device according to the first image and the second image, to determine a check result; and adjusting the on-board medical device in response to the check result indicating a failed accuracy check.

### RELATED APPLICATION

This application claims the benefit under 35 U.S.C. § 119(a) of the filing date of Chinese Patent Application No. 202411899857.1, filed in the Chinese Patent Office on Dec. 20, 2024. The disclosure of the foregoing application is herein incorporated by reference in its entirety.

### TECHNICAL FIELD

The present disclosure relates to the field of medical technologies, and in particular, to a monitoring method for an on-board medical device, a computer device, and a storage medium.

### BACKGROUND

With the development of medical technologies and society, on-board medical devices have emerged, such as on-board electronic computed tomography (CT) devices, on-board positron emission tomography (PET) devices, on-board single-photon emission computed tomography (SPECT) devices, on-board PET/CT devices, and on-board SPECT/CT devices. Normal operation of the on-board medical device has higher requirements for an environment and accuracy. Therefore, there is a need to monitor an environment and accuracy of the on-board medical device.

### SUMMARY

The present disclosure provides a monitoring method for an on-board medical device, a computer device, and a storage medium.

In a first aspect, the present disclosure provides a monitoring method for an on-board medical device, wherein the on-board medical device includes at least a first imaging assembly and a second imaging assembly, and the method includes:
acquiring environmental information of the on-board medical device;
in response to the environmental information exceeding a preset environmental threshold, controlling the first imaging assembly to image an imaging object to obtain a first image, and controlling the second imaging assembly to image the imaging object to obtain a second image;
performing an accuracy check on the on-board medical device according to the first image and the second image, to determine a check result, the check result being used to evaluate a relative position relationship between the first imaging assembly and the second imaging assembly; and
adjusting the on-board medical device in response to the check result indicating a failed accuracy check.

In an embodiment, the in response to the environmental information exceeding the preset environmental threshold, controlling the first imaging assembly to image the imaging object to obtain the first image, and controlling the second imaging assembly to image the imaging object to obtain the second image includes:
sending first prompt information in response to the environmental information exceeding the preset environmental threshold; and
in response to a control operation of a user based on the first prompt information, controlling the first imaging assembly to image the imaging object to obtain the first image, and controlling the second imaging assembly to image the imaging object to obtain the second image.

In an embodiment, the performing the accuracy check on the on-board medical device according to the first image and the second image, to determine the check result includes:
performing registration on the first image and the second image to obtain a registered image, the registered image being an image obtained by fusing the first image and the second image in a manner of aligning feature points or target regions; and
detecting quality of the registered image, to determine the check result.

In an embodiment, the detecting the quality of the registered image includes:
inputting the registered image into a pre-trained quality detection model, and detecting the inputted registered image through the quality detection model, to obtain the check result.

In an embodiment, the adjusting the on-board medical device in response to the check result indicating a failed accuracy check includes:
in response to the check result indicating a failed accuracy check:
adjusting an imaging parameter of the on-board medical device, or
adjusting relative positions of the first imaging assembly and the second imaging assembly.

In an embodiment, the performing the accuracy check on the on-board medical device according to the first image and the second image, to determine the check result includes:
performing registration on the first image and the second image to determine a to-be-measured registration coefficient; and
determining the check result according to the to-be-measured registration coefficient and a default registration coefficient.

In an embodiment, the adjusting the on-board medical device in response to the check result indicating a failed accuracy check includes:
replacing the default registration coefficient with the to-be-measured registration coefficient in response to the check result indicating a failed accuracy check; or sending adjustment information to the user to instruct the user to adjust relative positions of the first imaging assembly and the second imaging assembly.

In an embodiment, the determining the check result according to the to-be-measured registration coefficient and the default registration coefficient includes:
determining a difference between the to-be-measured registration coefficient and the default registration coefficient;
determining, in response to the difference being outside a preset difference range, that the check result indicates a failed accuracy check; and
determining, in response to the difference being within the preset difference range, that the check result indicates a passed accuracy check.

In an embodiment, the adjusting the on-board medical device in response to the check result indicating a failed accuracy check includes:
replacing the default registration coefficient with the to-be-measured registration coefficient in response to the difference being greater than or equal to a first preset difference threshold; and
sending adjustment information to the user in response to the difference being greater than or equal to a second preset difference threshold, to instruct the user to adjust a position of the on-board medical device; the second preset difference threshold being greater than the first preset difference threshold, and both the first preset difference threshold and the second preset difference threshold being outside the preset difference range.

In an embodiment, the method further includes:
cyclically performing the step of controlling the first imaging assembly to image an imaging object to obtain a first image, and controlling the second imaging assembly to image the imaging object to obtain a second image, the step of performing an accuracy check on the on-board medical device according to the first image and the second image, to determine a check result, and the step of adjusting the on-board medical device in response to the check result indicating a failed accuracy check, until the check result indicates a passed accuracy check or a number of times the accuracy check is performed being greater than or equal to a preset number-of-times threshold; and
in response to the number of times the accuracy check is performed being greater than or equal to the preset number-of-times threshold, sending second prompt information; the second prompt information being used to instruct the user to adjust the on-board medical device.

In an embodiment, the sending the first prompt information in response to the environmental information exceeding the preset environmental threshold includes:
determining an operating state of the on-board medical device;
sending the first prompt information in response to the environmental information exceeding the preset environmental threshold and the operating state being power on; and
sending, in response to the environmental information exceeding the preset environmental threshold and the operating state being power off, the first prompt information after the operating state of the on-board medical device transitions to power on.

In an embodiment, the performing registration on the first image and the second image to determine the to-be-measured registration coefficient includes:
extracting features of the first image and features of the second image respectively, the features of the first image being elements capable of identifying the first image, and the features of the second image being elements capable of identifying the second image;
performing matching on the features of the first image and the features of the second image; and
determining the to-be-measured registration coefficient according to matched features.

In an embodiment, the performing registration on the first image and the second image to determine the to-be-measured registration coefficient includes:
determining a grayscale value of the first image and a grayscale value of the second image respectively; and
determining the to-be-measured registration coefficient according to a similarity between the grayscale value of the first image and the grayscale value of the second image.

In an embodiment, the method further includes:
sending third prompt information in response to the check result indicating a passed accuracy check, the third prompt information being used to prompt that environmental information of the on-board medical device acquired by a user at a current moment does not affect accuracy of the on-board medical device.

In an embodiment, the first imaging assembly is PET or SPECT, and the second imaging assembly is CT.

In an embodiment, the environmental information includes at least one of temperature information, humidity information, air pressure information, and vibration information.

In an embodiment, the environmental information includes two or more of temperature information, humidity information, air pressure information, and vibration information; and
sending first prompt information in response to the environmental information exceeding the preset environmental threshold, including:
sending the first prompt information in response to any one of the environmental information exceeding the preset environmental threshold.

In a second aspect, an embodiment of the present disclosure provides a monitoring method for an on-board medical device, wherein the on-board medical device includes at least a first imaging assembly and a second imaging assembly, and the method includes:
acquiring vibration information of the on-board medical device at a current moment, and sending first prompt information in response to the vibration information exceeding a preset vibration threshold;
in response to a control operation of a user based on the first prompt information, controlling the first imaging assembly to image the imaging object to obtain the first image, and controlling the second imaging assembly to image the imaging object to obtain the second image;
performing registration on the first image and the second image to determine a to-be-measured registration coefficient; and determining a check result according to the to-be-measured registration coefficient and a default registration coefficient;
replacing the default registration coefficient with the to-be-measured registration coefficient in response to the check result indicating a failed accuracy check; or sending adjustment information to the user to instruct the user to adjust a position of the on-board medical device; and
cyclically performing the step of controlling the first imaging assembly to image an imaging object to obtain a first image, and controlling the second imaging assembly to image the imaging object to obtain a second image, the step of performing registration on the first image and the second image to determine a to-be-measured registration coefficient; and determining a check result according to the to-be-measured registration coefficient and a default registration coefficient, and the step of replacing the default registration coefficient with the to-be-measured registration coefficient in response to the check result indicating a failed accuracy check; or sending adjustment information to the user to instruct the user to adjust a position of the on-board medical device, until the check result indicates a passed accuracy check or a number of times the accuracy check is performed being greater than or equal to a preset number-of-times threshold.

In a third aspect, an embodiment of the present disclosure provides an on-board medical system, including an on-board medical device, a control device, and an environmental detection device that are communicatively connected with one another; wherein the environmental detection device is configured to detect environmental information of a mobile carrier where the on-board medical device is located, and send the environmental information to the control device; and the control device includes a memory and a processor, the memory storing a computer program, wherein the processor, when executing the computer program, implements steps of the methods provided in the first aspect and the second aspect above.

In a fourth aspect, an embodiment of the present disclosure provides a computer device, including a memory and a processor, the memory storing a computer program, wherein the processor, when executing the computer program, implements steps of the methods provided in the first aspect and the second aspect above.

In a fifth aspect, an embodiment of the present disclosure provides a computer-readable storage medium. When a computer program is executed by a processor, steps of the methods provided in the first aspect and the second aspect above are implemented.

In a sixth aspect, an embodiment of the present disclosure provides a computer program product, including a computer program. When the computer program is executed by a processor, steps of the methods provided in the first aspect and the second aspect above are implemented.

### BRIEF DESCRIPTION OF THE DRAWINGS

FIG. 1 is a diagram of an application environment of a monitoring method for an on-board medical device according to an embodiment;

FIG. 2 is a diagram flowchart of steps of the monitoring method for the on-board medical device according to an embodiment;

FIG. 3 is a diagram flowchart of steps of the monitoring method for the on-board medical device according to another embodiment;

FIG. 4 is a diagram flowchart of steps of the monitoring method for the on-board medical device according to another embodiment;

FIG. 5 is a diagram flowchart of steps of the monitoring method for the on-board medical device according to another embodiment;

FIG. 6 is a diagram flowchart of steps of the monitoring method for the on-board medical device according to another embodiment;

FIG. 7 is a diagram flowchart of steps of the monitoring method for the on-board medical device according to another embodiment;

FIG. 8 is a diagram flowchart of steps of the monitoring method for the on-board medical device according to another embodiment;

FIG. 9 is a diagram flowchart of steps of the monitoring method for the on-board medical device according to another embodiment; and

FIG. 10 is a schematic structural diagram of a monitoring apparatus for an on-board medical device according to an embodiment.

### DETAILED DESCRIPTION

In order to make the objectives, technical solutions, and advantages of the present disclosure more comprehensible, the present disclosure is further described in detail below with reference to the accompanying drawings and embodiments. It should be understood that specific embodiments described herein are only used to explain the present disclosure and are not intended to limit the present disclosure.

The serial numbers assigned to components herein, such as “first” and “second”, are used to distinguish described objects, but do not have any order or technical meaning.

First, before the technical solutions of the embodiments disclosed in the present disclosure are specifically introduced, the background or technical evolution context on which the embodiments of the present disclosure are based is introduced. With the development of medical technologies and society, mounting a medical device on a mobile carrier (such as a vehicle, an airplane, or a ship) can make it more convenient to move. Therefore, on-board medical devices have emerged. The on-board medical devices refer to medical devices mounted in mobile carriers such as vehicles, airplanes, and ships. The on-board medical devices may be, for example, on-board electronic CT devices, on-board PET devices, on-board SPECT devices, on-board PET/CT devices, on-board SPECT/CT devices, on-board radiotherapy devices, on-board digital subtraction angiography (DSA) devices, and the like. The on-board medical device may operate during movement of the mobile carrier. Normal operation of the on-board medical device has high requirements for an environment and accuracy. During movement and use of the on-board medical device, changes in environmental factors such as a temperature, humidity, air pressure, and vibration in the mobile carrier where the on-board medical device is located may affect accuracy of the on-board medical device and normal use of the on-board medical device. Therefore, there is a need to monitor an environment and accuracy of the on-board medical device. Generally, environmental factors such as an environment, humidity, and vibration of the mobile carrier where the on-board medical device is located are monitored. However, the conventional technology does not involve a technical solution of monitoring accuracy of an on-board medical device when environmental factors affect the accuracy of the on-board medical device. In this regard, the present disclosure provides a monitoring method for an on-board medical device that can monitor accuracy of the on-board medical device.

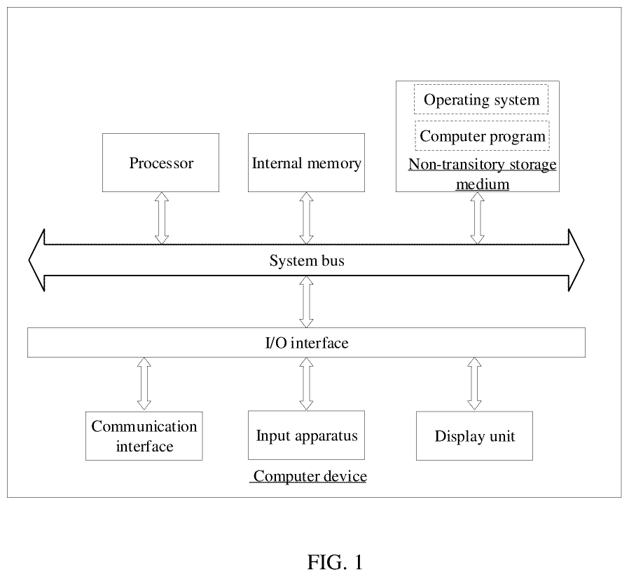

The monitoring method for the on-board medical device provided in the present disclosure may be applied to an on-board medical system. The on-board medical system includes an on-board medical device, a control device, and an environmental detection device. The control device is communicatively connected to both the on-board medical device and the environmental detection device. The on-board medical device and the control device may be arranged integrally or separately. The environmental detection device is configured to detect environmental information of a mobile carrier where the on-board medical device is located, and send the environmental information to the control device. The control device is configured to send a control signal to the on-board medical device based on the environmental information, to control the on-board medical device to operate. The control device may be an industrial computer, a laptop, a tablet computer, a control chip, or the like. An internal structure of the control device may be shown in FIG. 1. The control device may include a processor, a memory, a communication interface, a display screen, and an input apparatus connected through a system bus. The processor of the control device is configured to provide computing and control capabilities. The memory of the control device includes a non-transitory storage medium and an internal memory. The non-transitory storage medium stores an operating system and a computer program. The internal memory provides an environment for running of the operating system and the computer program in the non-transitory storage medium. The communication interface of the control device is configured to communicate with an external terminal in a wired or wireless manner. The wireless manner may be implemented through WIFI, a mobile cellular network, near field communication (NFC), or other technologies. The computer program is executed by the processor to implement a monitoring method for an on-board medical device. The display screen of the control device may be a liquid crystal display screen or an electronic ink display screen. The input apparatus of the control device may be a touchscreen covering the display screen, or may be a key, a trackball, or a touchpad disposed on a housing of the control device, or may be an external keyboard, a touchpad, a mouse, or the like.

Those skilled in the art may understand that, in the structure shown in FIG. 1, only a block diagram of a partial structure related to the solution of the present disclosure is shown, which does not constitute a limitation on the computer device to which the solution of the present disclosure is applied. Specifically, the computer device may include more or fewer components than those shown in the figure, or some components may be combined, or a different component deployment may be used.

The technical solutions of the present disclosure and how the technical solutions of the present disclosure solve the technical problems are described below with specific embodiments.

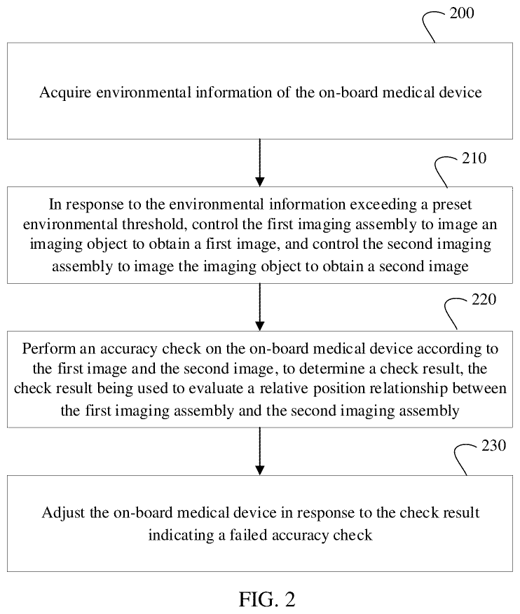

In an embodiment, as shown in FIG. 2, a monitoring method for an on-board medical device is provided. This embodiment is illustrated based on an example in which the method is applied to a control device in an on-board medical system. The on-board medical device may include at least two imaging assemblies, which are a first imaging assembly and a second imaging assembly respectively. For example, in terms of an on-board PET/CT device, the first imaging assembly is PET, and the second imaging assembly is CT. In terms of an on-board SPECT/CT device, the first imaging assembly is SPECT, and the second imaging assembly is CT. In this embodiment, the method includes the following steps.

In step 200, environmental information of the on-board medical device is acquired.

The environmental information may include various environmental information of a mobile carrier where the on-board medical device is located. The environmental information may include at least one of temperature information, humidity information, air pressure information, and vibration information. The environmental information may be acquired in real time or periodically by using an environmental detection device arranged in the mobile carrier or arranged on the on-board medical device and transmitted to the control device. The acquiring the environmental information of the on-board medical device may be acquiring environmental information of the on-board medical device at a current moment.

In an optional embodiment, the environmental detection device may include a temperature sensor, a humidity sensor, a barometer, and a vibration sensor. The temperature sensor may be employed to collect a temperature value inside the mobile carrier in real time. The humidity sensor may be employed to collect a humidity value inside the mobile carrier in real time. The barometer may be employed to collect an air pressure value inside the mobile carrier in real time. The vibration sensor may be employed to collect a vibration value inside the mobile carrier in real time.

In step 210, in response to the environmental information exceeding a preset environmental threshold, the first imaging assembly is controlled to image an imaging object to obtain a first image, and the second imaging assembly is controlled to image the imaging object to obtain a second image.

The preset environmental threshold corresponds to the environmental information. The preset environmental threshold may include at least one of a preset temperature threshold, a preset humidity threshold, a preset air pressure threshold, a preset vibration threshold, and the like.

After acquiring the environmental information of the on-board medical device at the current moment, the control device may compare the environmental information with the preset environmental threshold, and may obtain a comparison result. That is, a temperature value in the environmental information may be compared with the preset temperature threshold, a humidity value in the environmental information may be compared with the preset humidity threshold, an air pressure value in the environmental information may be compared with the preset air pressure threshold, and a vibration value in the environmental information may be compared with the preset vibration threshold. According to the comparison result, it may be determined whether the environmental information exceeds the preset environmental threshold. In response to the environmental information exceeding the preset environmental threshold, a first control signal is sent to the first imaging assembly in the on-board medical device, to control the first imaging assembly to scan and image the imaging object to obtain the first image, and a second control signal is sent to the second imaging assembly in the on-board medical device, to control the second imaging assembly to scan and image the imaging object to obtain the second image. Types of the first image and the second image correspond to types of the first imaging assembly and the second imaging assembly. If the first imaging assembly is PET and the second imaging assembly is CT, the first image is a PET image and the second image is a CT image. If the first imaging assembly is SPECT and the second imaging assembly is CT, the first image is a SPECT image and the second image is a CT image.

A control operation may be a pressing or toggling operation performed by a user on a control button arranged on the on-board medical device or a clicking or dragging operation performed by the user on a control widget on a display interface of the control device.

In an optional embodiment, the in response to the environmental information exceeding the preset environmental threshold, controlling the first imaging assembly to image the imaging object to obtain the first image, and controlling the second imaging assembly to image the imaging object to obtain the second image includes: sending first prompt information in response to the environmental information exceeding the preset environmental threshold, and in response to a control operation of a user based on the first prompt information, controlling the first imaging assembly to image the imaging object to obtain the first image, and controlling the second imaging assembly to image the imaging object to obtain the second image.

The first prompt information is sent in response to the comparison result being the environmental information exceeding the preset environmental threshold. The first prompt information may be a sound prompt or a visual prompt, such as a prompt bell, a prompt light, a prompt box popped up on the display interface of the control device and/or a display interface of the on-board medical device, or a voice prompt. Optionally, the first prompt information sent by the control device may be persistent. In the present application, sending prompt information refers to generating and issuing prompt information.

When the environmental information includes multiple types of information, the first prompt information is sent in response to any environmental information exceeding the preset environmental threshold. In response to the environmental information including a temperature value, a humidity value, and a vibration value, the first prompt information is sent if any one of the temperature value, the humidity value, and the vibration value exceeds the corresponding preset environmental threshold.

The first prompt information is used to prompt the user to perform a series of operations including a control operation, which will be described in detail later.

The imaging object may be an imaging phantom, or may be a plant, an animal, a human body, or the like. After receiving the first prompt information, the user places the imaging object at a target position corresponding to the first imaging assembly. The target position corresponding to the first imaging assembly refers to a position in a same axial direction as the first imaging assembly. After placing the imaging object at the target position corresponding to the first imaging assembly, the user performs a control operation based on the first prompt information. After receiving the control operation of the user, the control device sends the first control signal to the first imaging assembly in the on-board medical device, to control the first imaging assembly to scan and image the imaging object to obtain the first image.

After receiving the first prompt information, the user places the imaging object at a target position corresponding to the second imaging assembly. The target position corresponding to the second imaging assembly refers to a position in a same axial direction as the second imaging assembly. After placing the imaging object at the target position corresponding to the second imaging assembly, the user performs a control operation based on the first prompt information. After receiving the control operation of the user, the control device sends the first control signal to the first imaging assembly in the on-board medical device, to control the first imaging assembly to scan and image the imaging object to obtain the first image, and sends the second control signal to the second imaging assembly in the on-board medical device, to control the second imaging assembly to scan and image the imaging object to obtain the second image.

In step 220, an accuracy check is performed on the on-board medical device according to the first image and the second image, to determine a check result, the check result being used to evaluate a relative position relationship between the first imaging assembly and the second imaging assembly.

After obtaining the first image and the second image, the control device performs an accuracy check on the on-board medical device according to the first image and the second image, that is, determines whether positions of the first imaging assembly and the second imaging assembly in the on-board medical device meet a preset condition, to determine the check result. The check result includes a passed accuracy check or a failed accuracy check. The passed accuracy check means that a relative position relationship between the first imaging assembly and the second imaging assembly in the on-board medical device meets the preset condition. The failed accuracy check means that the relative position relationship between the first imaging assembly and the second imaging assembly in the on-board medical device fails to meet the preset condition. In this embodiment, a specific method for determining the check result according to the first image and the second image is not limited, as long as the function thereof can be implemented.

In an optional embodiment, after obtaining the first image and the second image, the control device performs registration on the first image and the second image to obtain a registered image, and detects quality of the registered image, to determine the check result according to a detection result. If the quality of the registered image meets a preset quality condition, it is determined that the check result indicates a passed accuracy check. If the quality of the registered image does not meet the preset quality condition, it is determined that the check result indicates a failed accuracy check. The registered image is an image obtained by fusing the first image and the second image in a manner of aligning feature points or target regions. The preset quality condition may be that a degree of overlap between the feature points or target regions of the first image and the second image reaches a preset standard. A method for detecting the quality of the registered image may include: inputting the registered image into a pre-trained quality detection model, and detecting the inputted registered image through the quality detection model, to obtain the detection result.

In step 230, the on-board medical device is adjusted in response to the check result indicating a failed accuracy check.

The on-board medical device is adjusted in response to the check result of the accuracy check performed by the control device on the on-board medical device according to the first image and the second image indicating a failed accuracy check, to ensure accuracy of the on-board medical device. The adjusting the on-board medical device may include adjusting an imaging parameter of the on-board medical device or adjusting relative positions of the first imaging assembly and the second imaging assembly. Adjusting the imaging parameter(s) of the on-board medical device refers to adjusting the operating parameters of the first imaging assembly and the second imaging assembly to compensate for imaging deviations caused by environmental factors (such as vibration, temperature and humidity changes), thereby ensuring that the registration accuracy of the first image and the second image as well as the overall detection accuracy of the equipment meet the required standards. Examples of the imaging parameter(s) of on-board medical device include, but not limited to: tube voltage, tube current, and scan slice thickness of the CT imaging assembly; detector sensitivity threshold and scanning time window of the PET imaging assembly; collimator type and filter function of the SPECT imaging assembly, or the like. Adjusting relative positions of the first imaging assembly and the second imaging assembly refers to, for example, adjusting positions of the first imaging assembly, or adjusting the position of the second imaging assembly, or simultaneously adjusting the positions of both the first and second imaging assemblies to cause the positions of the first imaging assembly, the second imaging assembly, and the scanning bed to be in a same axial direction. For another example, adjusting relative positions of the first imaging assembly and the second imaging assembly refers to adjusting the position of the scanning bed, as the scanning bed can be controlled independently. In this embodiment, a specific method for adjusting the on-board medical device is not limited, as long as the function thereof can be implemented.

In an optional embodiment, third prompt information is sent in response to the control device determining that the check result indicates a passed accuracy check. The third prompt information is used to prompt that the environmental information of the on-board medical device acquired by the user at the current moment does not affect the accuracy of the on-board medical device. That is, the on-board medical device may be used normally. In an optional embodiment, in response to the control device determining that the check result indicates a passed accuracy check, the first prompt information may be stopped, and the third prompt information is sent. The third prompt information may be a sound prompt or a visual prompt, such as a prompt bell, a prompt light, a prompt box popped up on the display interface of the control device and/or the display interface of the on-board medical device, or a voice prompt. The first prompt information may also be sent in response to the control device determining that environmental information of the on-board medical device acquired at next moment still exceeds the preset environmental threshold. That is, a passed accuracy check on the on-board medical device at a current moment does not mean a passed accuracy check on the on-board medical device at next moment.

In an optional embodiment, the control device repeats the above operations at a predetermined interval. That is, the control device acquires environmental information of the on-board medical device at a current moment at a predetermined interval, thereby determining a check result.

In the monitoring method for the on-board medical device provided in the embodiments of the present disclosure, environmental information of the on-board medical device at a current moment is acquired, first prompt information is sent in response to the environmental information exceeding a preset environmental threshold, in response to a control operation of a user based on the first prompt information, the first imaging assembly in the on-board medical device is controlled to image an imaging object to obtain a first image, and the second imaging assembly is controlled to image the imaging object to obtain a second image, an accuracy check is performed on the on-board medical device according to the first image and the second image, to determine a check result, and the on-board medical device is adjusted in response to the check result indicating a failed accuracy check. In this embodiment, through the acquired environmental information of the on-board medical device at the current moment, monitoring of the environmental information of the on-board medical device at the current moment can be implemented, and an accuracy check on the on-board medical device at the current moment can also be implemented when the environmental information exceeds the preset environmental threshold, that is, the accuracy of the on-board medical device may be affected, thereby preventing obtaining of an image whose quality does not meet the preset quality condition by the user by using the on-board medical device not passing the accuracy check. Moreover, in response to determining that the on-board medical device does not pass the accuracy check, the on-board medical device may be adjusted, to ensure the accuracy of the on-board medical device, so that the monitoring method for the on-board medical device provided in the present disclosure has higher reliability and practicality.

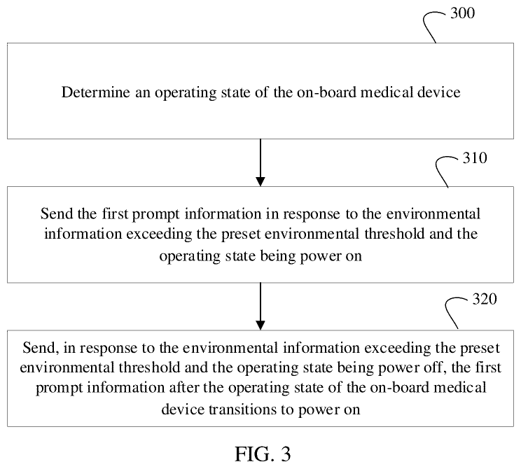

In an embodiment, as shown in FIG. 3, an implementation of sending the first prompt information in response to the environmental information exceeding the preset environmental threshold is involved. The implementation includes the following steps.

In step 300, an operating state of the on-board medical device is determined.

The operating state of the on-board medical device may include the operating state of the on-board medical device being power on or the operating state of the on-board medical device being power off. The operating state “power on” indicates that the on-board medical device is in an operating state, and the operating state “power off” indicates that the on-board medical device is not in the operating state, which includes, for example, a powered-but-standby state. In this embodiment, a specific method for determining the operating state of the on-board medical device is not limited, as long as the function thereof can be implemented.

In an optional embodiment, an operating state indicator is arranged on the on-board medical device, and the control device may determine the operating state of the on-board medical device by monitoring a state of the indicator. If it is detected that the indicator is on, it is determined that the operating state of the on-board medical device is power on. If it is detected that the indicator is off, it is determined that the operating state of the on-board medical device is power off.

In another optional embodiment, the control device may determine the operating state of the on-board medical device by detecting power supplied to the on-board medical device. If it is detected that the power supplied to the on-board medical device is greater than a preset power threshold, it is determined that the operating state of the on-board medical device is power on. If it is detected that the power supplied to the on-board medical device is less than or equal to the preset power threshold, it is determined that the operating state of the on-board medical device is power off.

In step 310, the first prompt information is sent in response to the environmental information exceeding the preset environmental threshold and the operating state being power on.

After determining the operating state of the on-board medical device, the control device sends the first prompt information when determining that the environmental information of the on-board medical device at the current moment exceeds the preset environmental threshold and in response to the operating state of the on-board medical device being power on. That is, in response to the operating state of the on-board medical device being power on, the first prompt information is sent as long as it is determined that the environmental information of the on-board medical device at the current moment exceeds the preset environmental threshold.

In step 320, in response to the environmental information exceeding the preset environmental threshold and the operating state being power off, the first prompt information is sent after the operating state of the on-board medical device transitions to power on.

After determining the operating state of the on-board medical device, the control device, when determining that the environmental information of the on-board medical device at the current moment exceeds the preset environmental threshold and in response to the operating state of the on-board medical device being power off, continuously monitors the operating state of the on-board medical device in real time, and sends the first prompt information after monitoring that the operating state of the on-board medical device transitions to power on. That is, in response to the operating state of the on-board medical device being power off and a determination that the environmental information at the current moment exceeds the preset environmental threshold, after the operating state of the on-board medical device transitions to power on, the first prompt information may be sent regardless of whether the environmental information of the on-board medical device exceeds the preset environmental threshold after the operating state of the on-board medical device transitions to power on.

In this embodiment, by determining the operating state of the on-board medical device, the first prompt information is sent in response to the environmental information exceeding the preset environmental threshold and the operating state being power on. In response to the environmental information exceeding the preset environmental threshold and the operating state being power off, the first prompt information is sent after the operating state of the on-board medical device transitions to power on. In this embodiment, in response to the operating state of the on-board medical device being power on, the first prompt information may be sent in real time, to prompt, in a timely manner, the user to perform an accuracy check on the on-board medical device to ensure the accuracy of the on-board medical device. After the operating state of the on-board medical device transitions from power off to power on, the first prompt information may be sent regardless of whether the environmental information of the on-board medical device exceeds the preset environmental threshold after the operating state of the on-board medical device transitions to power on, to prompt that the operating state of the on-board medical device being power off and the environmental information exceeding the preset environmental threshold affect the accuracy of the on-board medical device. In this case, even if the environmental information does not exceed the preset environmental threshold after the operating state of the on-board medical device transitions to power on, there is still a need to perform the accuracy check on the on-board medical device, which can prevent use of the on-board medical device not passing the accuracy check by the user, so as to ensure the accuracy of the on-board medical device used, thereby enabling higher reliability of the monitoring method for the on-board medical device.

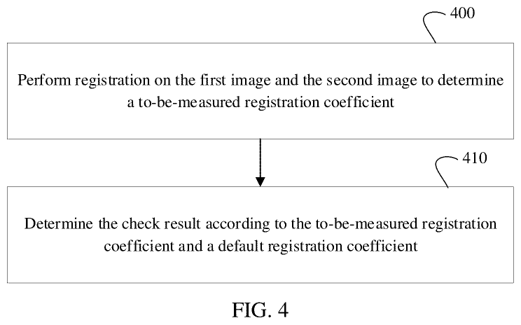

In an embodiment, as shown in FIG. 4, an implementation of determining the check result according to the first image and the second image is involved. The implementation includes the following steps.

In step 400, registration is performed on the first image and the second image to determine a to-be-measured registration coefficient.

After obtaining the first image and the second image, the control device performs registration on the first image and the second image, and may obtain the to-be-measured registration coefficient between the first image and the second image. In this embodiment, a specific method for performing registration on the first image and the second image to determine the to-be-measured registration coefficient is not limited, as long as the function thereof can be implemented.

In an optional embodiment, the method for performing registration on the first image and the second image may be a feature-based registration method. Specifically, features of the first image and features of the second image are extracted respectively. Matching is performed on the features of the first image and the features of the second image. The to-be-measured registration coefficient is determined according to matched features. Features of an image refer to elements capable of identifying the image, for example, a structure of an imaging object in the image, a texture feature of the image, positions of key points, and the like. The to-be-measured registration coefficient may be a registration matrix. The registration matrix includes a translation matrix and a rotation matrix. The method for performing registration on the first image and the second image may be a grayscale-based registration method. Specifically, a grayscale value of the first image and a grayscale value of the second image are determined respectively, and the to-be-measured registration coefficient is determined according to a similarity between the grayscale value of the first image and the grayscale value of the second image.

In step 410, the check result is determined according to the to-be-measured registration coefficient and a default registration coefficient.

The default registration coefficient may be a registration coefficient preset by the user or a registration coefficient obtained after previous use of the on-board medical device for imaging, which is not limited in this embodiment.

After obtaining the to-be-measured registration coefficient, the control device compares the to-be-measured registration coefficient with the default registration coefficient, to determine the check result. Specifically, the control device may determine a difference between the to-be-measured registration coefficient and the default registration coefficient, determine, in response to the difference being within a preset difference range, that the check result indicates a passed accuracy check, and determine, in response to the difference being outside the preset difference range, that the check result indicates a failed accuracy check.

In this embodiment, the to-be-measured registration coefficient is determined by performing registration on the first image and the second image, and the check result is determined according to the to-be-measured registration coefficient and the default registration coefficient. In this way, the method for determining the check result is quick and easy to implement, which can improve efficiency of determination of the check result, so that the on-board medical device can be adjusted in time when the check result indicates a failed accuracy check, to ensure that the on-board medical device can be used normally.

In an embodiment, an implementation of adjusting the on-board medical device in response to the check result indicating a failed accuracy check is involved. The implementation includes:
replacing the default registration coefficient with the to-be-measured registration coefficient in response to the check result indicating a failed accuracy check, or sending adjustment information to the user to instruct the user to adjust a position of the on-board medical device.

The control device may replace the default registration coefficient with the to-be-measured registration coefficient in response to determining that the check result indicates a failed accuracy check, to implement adjustment of the on-board medical device. The control device may alternatively send adjustment information to the user in response to determining that the check result indicates a failed accuracy check, and the user adjusts the on-board medical device based on the adjustment information, to implement adjustment of the on-board medical device. After adjusting the position of the on-board medical device, the user may send confirmation information to the control device, so that the control device receives information indicating that the user has adjusted the position of the on-board medical device.

In an optional embodiment, when the on-board medical device includes the first imaging assembly and the second imaging assembly, the user adjusting the position of the on-board medical device may be adjusting positions of the first imaging assembly, the second imaging assembly, and a scanning bed, to cause the positions of the first imaging assembly, the second imaging assembly, and the scanning bed to be in a same axial direction.

In this embodiment, two implementations of adjusting the on-board medical device in response to the check result indicating a failed accuracy check are provided. In one implementation, the default registration coefficient is replaced with the to-be-measured registration coefficient. In the other implementation, adjustment information is sent to the user to instruct the user to adjust the position of the on-board medical device. The user may make a selection according to an actual application situation, which can improve reliability and practicality of the monitoring method for the on-board medical device.

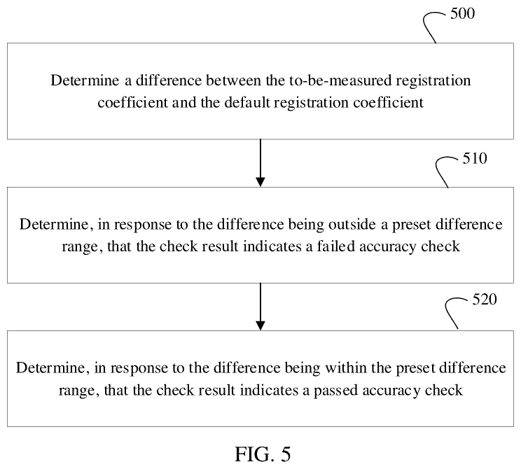

In an embodiment, as shown in FIG. 5, an implementation of determining the check result according to the to-be-measured registration coefficient and the default registration coefficient is involved. The implementation includes the following steps.

In step 500, a difference between the to-be-measured registration coefficient and the default registration coefficient is determined.

After determining the to-be-measured registration coefficient, the control device calculates the difference between the to-be-measured registration coefficient and the default registration coefficient.

In step 510, it is determined, in response to the difference being outside a preset difference range, that the check result indicates a failed accuracy check.

The preset difference range may be set by the user according to actual experience and be stored in the control device. After determining the difference between the to-be-measured registration coefficient and the default registration coefficient, the control device compares the difference with the preset difference range, and determines, in response to determining that the difference is outside the preset difference range, that the check result indicates a failed accuracy check.

In step 520, it is determined, in response to the difference being within the preset difference range, that the check result indicates a passed accuracy check.

The control device determines, in response to determining that the difference between the to-be-measured registration coefficient and the default registration coefficient is within the preset difference range, that the check result indicates a passed accuracy check.

In this embodiment, the check result is determined by determining whether the difference between the to-be-measured registration coefficient and the default registration coefficient is within the preset difference range. In this way, the method for determining the check result is quick and easy to implement, which can improve efficiency of determination of the check result, so that the on-board medical device can be adjusted in time when the check result indicates a failed accuracy check, so as to ensure that the on-board medical device can be used normally.

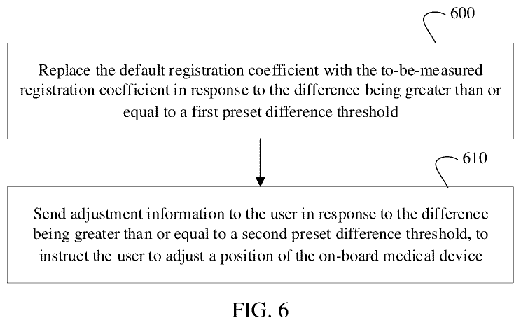

When the check result is determined according to the difference between the to-be-measured registration coefficient and the default registration coefficient, as shown in FIG. 6, another implementation of adjusting the on-board medical device in response to the check result indicating a failed accuracy check is involved. The implementation includes the following steps.

In step 600, the default registration coefficient is replaced with the to-be-measured registration coefficient in response to the difference being greater than or equal to a first preset difference threshold.

The first preset difference threshold may be set by the user according to actual experience and be stored in the control device. After obtaining the difference between the to-be-measured registration coefficient and the default registration coefficient, the control device compares the difference with the first preset difference threshold, and if determining that the difference is greater than or equal to the first preset difference threshold, implements adjustment of the on-board medical device by replacing the default registration coefficient with the to-be-measured registration coefficient.

In step 610, adjustment information is sent to the user in response to the difference being greater than or equal to a second preset difference threshold, to instruct the user to adjust a position of the on-board medical device, the second preset difference threshold being greater than the first preset difference threshold, and both the first preset difference threshold and the second preset difference threshold being outside the preset difference range.

The second preset difference threshold may also be set by the user according to actual experience and be stored in the control device. The second preset difference threshold is greater than the first preset difference threshold, and both the first preset difference threshold and the second preset difference threshold are outside the preset difference range. After comparing the difference between the to-be-measured registration coefficient and the default registration coefficient with the second preset difference threshold, the control device implements adjustment of the on-board medical device by sending adjustment information to the user to instruct the user to adjust the position of the on-board medical device if determining that the difference is greater than or equal to the second preset difference threshold.

In this embodiment, a specific method used when the on-board medical device is adjusted is determined by comparing the difference with the first preset difference threshold and the second preset difference threshold respectively. In this way, the specific method for adjusting the on-board medical device under different difference conditions can be clarified, so that the monitoring method for the on-board medical device has higher reliability.

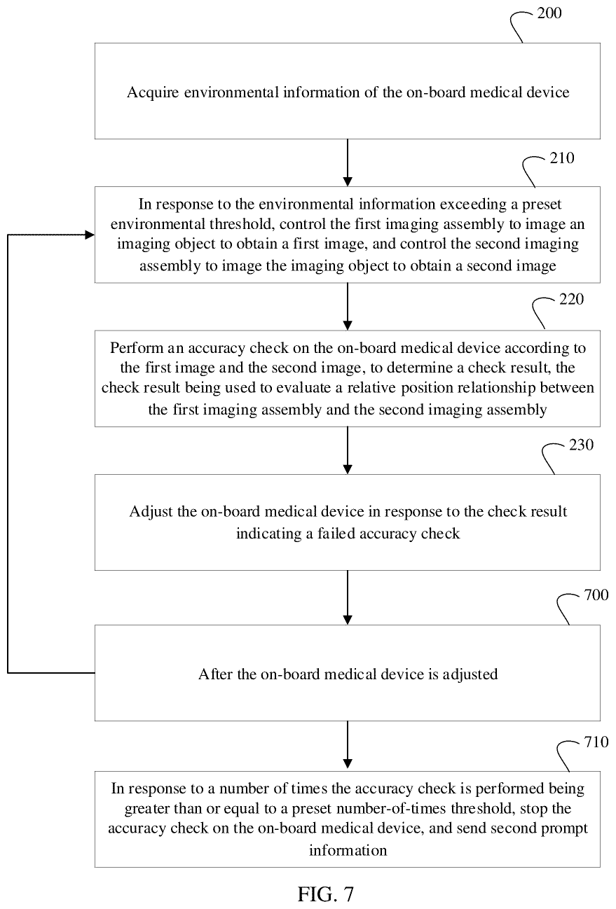

In an embodiment, as shown in FIG. 7, the method further includes the following steps.

In step 700, after the on-board medical device is adjusted, the step of controlling the first imaging assembly to image an imaging object to obtain a first image, and controlling the second imaging assembly to image the imaging object to obtain a second image, the step of performing an accuracy check on the on-board medical device according to the first image and the second image, to determine a check result, and the step of adjusting the on-board medical device in response to the check result indicating a failed accuracy check is cyclically performed, until the check result indicates a passed accuracy check or a number of times the accuracy check is performed being greater than or equal to a preset number-of-times threshold.

After the check result indicates a failed accuracy check and the control device adjusts the on-board medical device, step 210 of controlling the first imaging assembly to image an imaging object to obtain a first image, and controlling the second imaging assembly to image the imaging object to obtain a second image, step 220 and step 230 are cyclically performed, until the check result is indicates passed accuracy check or a number of times the accuracy check is performed being greater than or equal to a preset number-of-times threshold. That is, after the on-board medical device is adjusted, the accuracy check is performed on the adjusted on-board medical device again.

In step 710, in response to a number of times the accuracy check is performed being greater than or equal to a preset number-of-times threshold, the accuracy check on the on-board medical device is stopped, and second prompt information is sent, the second prompt information being used to instruct the user to adjust the on-board medical device.

The preset number-of-times threshold may be preset by the user and be stored in the control device. When step 210 of controlling the first imaging assembly to image an imaging object to obtain a first image, and controlling the second imaging assembly to image the imaging object to obtain a second image, step 220 and step 230 are returned to perform after the control device adjusts the on-board medical device, a number of times the accuracy check is performed on the on-board medical device currently may be determined, and in response to the number of times the accuracy check is performed being greater than or equal to the preset number-of-times threshold, the step of performing the accuracy check on the on-board medical device again may be stopped, and the second prompt information is sent. The second prompt information is used to instruct the user to adjust the on-board medical device. That is, after the accuracy check is performed on the on-board medical device multiple times, the accuracy check on the on-board medical device is still a failed accuracy check, indicating that the current method for adjusting the on-board medical device cannot ensure the accuracy of the on-board medical device. Therefore, the user is required to intervene to adjust the on-board medical device. The second prompt information may be a prompt bell or a prompt light, or may be a prompt box popped up on the display interface of the control device and/or the display interface of the on-board medical device, or may be a voice prompt.

In this embodiment, after the on-board medical device is adjusted, the accuracy check may be performed on the adjusted on-board medical device again, which can verify an adjustment result to ensure accuracy of the adjusted on-board medical device. Moreover, in response to a number of times the accuracy check is performed on the on-board medical device being greater than or equal to a preset number-of-times threshold, the accuracy check on the on-board medical device may be stopped, and the second prompt information is sent, to instruct the user to adjust the on-board medical device. In this way, repeated accuracy checks on the on-board medical device when the accuracy of the on-board medical device cannot be ensured by using the current method for adjusting the on-board medical device can be prevented, thereby reducing a waste of resources. By sending the second prompt information, the user can be instructed to adjust the on-board medical device so that the on-board medical device can be used normally as soon as possible.

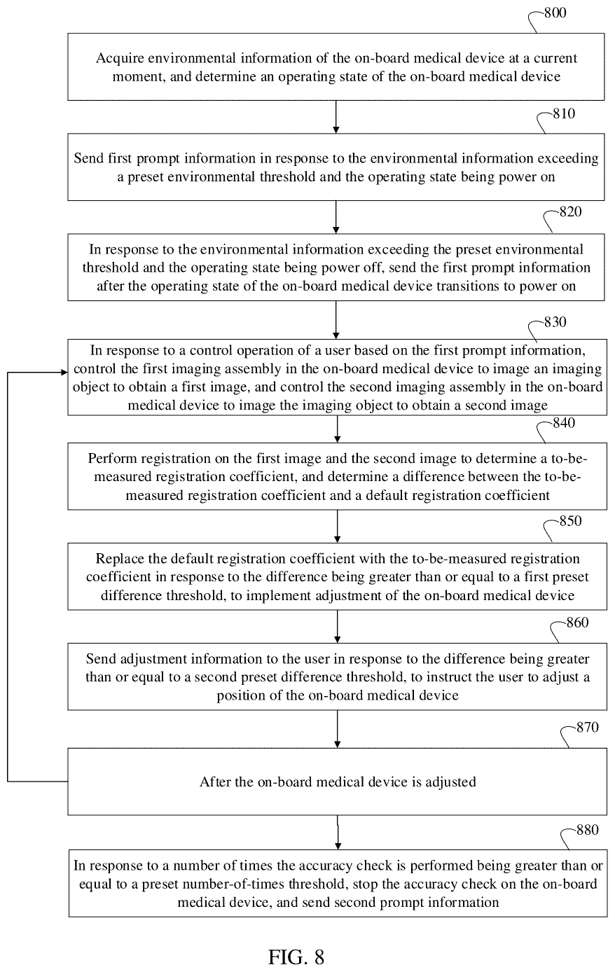

Referring to FIG. 8, an embodiment of the present disclosure provides a monitoring method for an on-board medical device. The on-board medical device includes at least a first imaging assembly and a second imaging assembly. The method includes the following steps.

In step 800, environmental information of the on-board medical device at a current moment is acquired, and an operating state of the on-board medical device is determined.

In step 810, first prompt information is sent in response to the environmental information exceeding a preset environmental threshold and the operating state being power on.

In step 820, in response to the environmental information exceeding the preset environmental threshold and the operating state being power off, the first prompt information is sent after the operating state of the on-board medical device transitions to power on.

In step 830, in response to a control operation of a user based on the first prompt information, the first imaging assembly in the on-board medical device is controlled to image an imaging object to obtain a first image, and the second imaging assembly in the on-board medical device is controlled to image the imaging object to obtain a second image.

In step 840, registration is performed on the first image and the second image to determine a to-be-measured registration coefficient, and a difference between the to-be-measured registration coefficient and a default registration coefficient is determined.

In step 850, the default registration coefficient is replaced with the to-be-measured registration coefficient in response to the difference being greater than or equal to a first preset difference threshold, to implement adjustment of the on-board medical device.

In step 860, adjustment information is sent to the user in response to the difference being greater than or equal to a second preset difference threshold, to instruct the user to adjust a position of the on-board medical device.

In step 870, after the on-board medical device is adjusted, step 830 of controlling the first imaging assembly in the on-board medical device to image an imaging object to obtain a first image, and controlling the second imaging assembly in the on-board medical device to image the imaging object to obtain a second image and step 840 to step 860 are cyclically performed, until the check result indicates a passed accuracy check or a number of times the accuracy check is performed being greater than or equal to a preset number-of-times threshold.

In step 880, in response to the number of times the accuracy check is performed being greater than or equal to the preset number-of-times threshold, the accuracy check on the on-board medical device is stopped, and second prompt information is sent, the second prompt information being used to instruct the user to adjust the on-board medical device.

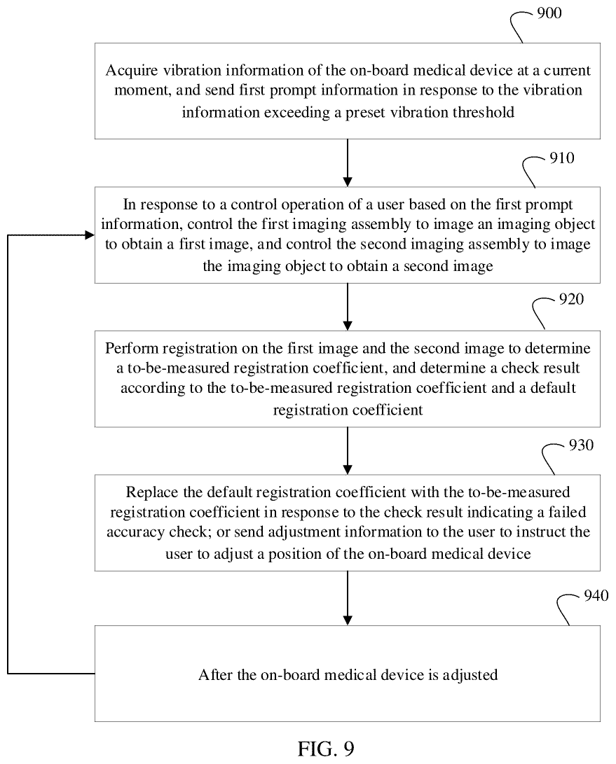

In an embodiment, as shown in FIG. 9, a monitoring method for an on-board medical device is provided. The on-board medical device includes at least a first imaging assembly and a second imaging assembly. This embodiment is illustrated based on an example in which the method is applied to a control device in an on-board medical system. In this embodiment, the method includes the following steps.

In step 900, vibration information of the on-board medical device at a current moment is acquired, and first prompt information is sent in response to the vibration information exceeding a preset vibration threshold.

In step 910, in response to a control operation of a user based on the first prompt information, the first imaging assembly is controlled to image an imaging object to obtain a first image, and the second imaging assembly is controlled to image the imaging object to obtain a second image.

In step 920, registration is performed on the first image and the second image to determine a to-be-measured registration coefficient, and a check result is determined according to the to-be-measured registration coefficient and a default registration coefficient.

In step 930, the default registration coefficient is replaced with the to-be-measured registration coefficient in response to the check result indicating a failed accuracy check, or adjustment information is sent to the user to instruct the user to adjust a position of the on-board medical device.

In step 940, after the on-board medical device is adjusted, step 910 of controlling the first imaging assembly to image an imaging object to obtain a first image, and controlling the second imaging assembly to image the imaging object to obtain a second image and step 920 to step 930 are cyclically performed, until the check result indicates a passed accuracy check or a number of times the accuracy check is performed being greater than or equal to a preset number-of-times threshold.

For specific description of the monitoring method in this embodiment, refer to the specific description in the foregoing embodiments. Details are not described herein again.

It should be understood that, although the steps in the flowchart as referred to in the embodiments as described above are shown in sequence as indicated by the arrows, the steps are not necessarily performed in the order indicated by the arrows. Unless otherwise clearly specified herein, the steps are performed without any strict sequence limitation, and the steps may be performed in other orders. In addition, at least some steps in the flowchart as referred to in the embodiments as described above may include a plurality of steps or a plurality of stages, and such steps or stages are not necessarily performed at a same moment, and may be performed at different moments. The steps or stages are not necessarily performed in sequence, and the steps or stages and at least some of other steps or steps or stages of other steps may be performed in turn or alternately.

Based on a same invention concept, the embodiments of the present disclosure further provide a monitoring apparatus for an on-board medical device configured to implement the monitoring method for the on-board medical device as referred to above. The implementation solution provided by the apparatus to the problem is similar to the implementation solution described in the above method. Therefore, for specific limitations in one or more embodiments of the monitoring apparatus for the on-board medical device provided below, refer to the limitations on the monitoring method for the on-board medical device above. Details are not described herein again.

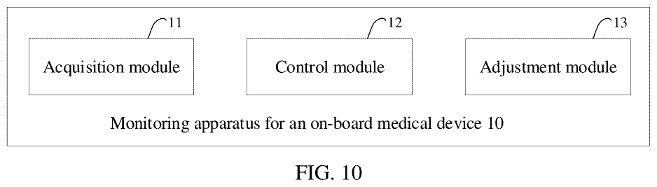

In an embodiment, as shown in FIG. 10, a monitoring apparatus for an on-board medical device 10 is provided. The on-board medical device includes at least a first imaging assembly and a second imaging assembly. The apparatus includes: an acquisition module 11, a control module 12, and an adjustment module 13.

The acquisition module 11 is configured to acquire environmental information of the on-board medical device.

The control module 12 is configured to, in response to the environmental information exceeding the preset environmental threshold, control the first imaging assembly to image an imaging object to obtain a first image, and control the second imaging assembly to image the imaging object to obtain a second image.

The adjustment module 13 is configured to perform an accuracy check on the on-board medical device according to the first image and the second image, determine a check result, and adjust the on-board medical device in response to the check result indicating a failed accuracy check, the check result being used to evaluate a relative position relationship between the first imaging assembly and the second imaging assembly.

The modules in the foregoing monitoring apparatus for the on-board medical device may be implemented entirely or partially by software, hardware, or a combination thereof. The above modules may be built in or independent of a processor of a computer device in a hardware form, or may be stored in a memory of the computer device in a software form, to facilitate the processor to invoke and perform operations corresponding to the above modules.

The features described in the embodiments of the foregoing monitoring method for the on-board medical device are applicable to the embodiments of the monitoring apparatus for the on-board medical device. For various implementations of the embodiments of the monitoring apparatus for the on-board medical device, refer to the relevant description in the embodiments of the foregoing monitoring method for the on-board medical device. Details are not described herein again.

In an embodiment, an on-board medical system is provided, including an on-board medical device, a control device, and an environmental detection device that are communicatively connected with one another. The environmental detection device is configured to detect environmental information of a mobile carrier where the on-board medical device is located, and send the environmental information to the control device. The control device includes a memory and a processor. The memory stores a computer program. The processor performs steps of the monitoring method for the on-board medical device above.

The features described in the embodiments of the foregoing monitoring method for the on-board medical device are applicable to the embodiments of the on-board medical system. For various implementations of the embodiments of the on-board medical system, refer to the relevant description in the embodiments of the foregoing monitoring method for the on-board medical device. Details are not described herein again.

In an embodiment, a computer device is provided. The computer device may be a control device. A diagram of an internal structure thereof may be shown in FIG. 1.

In an embodiment, a computer device is provided, including a memory and a processor. The memory stores a computer program. The processor, when executing the computer program, implements the following steps:
acquiring environmental information of the on-board medical device;
in response to the environmental information exceeding a preset environmental threshold, controlling the first imaging assembly to image an imaging object to obtain a first image, and controlling the second imaging assembly to image the imaging object to obtain a second image;
performing an accuracy check on the on-board medical device according to the first image and the second image, to determine a check result, the check result being used to evaluate a relative position relationship between the first imaging assembly and the second imaging assembly; and
adjusting the on-board medical device in response to the check result indicating a failed accuracy check.

In an embodiment, the processor, when executing the computer program, further implements the following steps: sending first prompt information in response to the environmental information exceeding the preset environmental threshold, and in response to a control operation of a user based on the first prompt information, controlling the first imaging assembly to image an imaging object to obtain a first image, and controlling the second imaging assembly to image the imaging object to obtain a second image.

In an embodiment, the processor, when executing the computer program, further implements the following steps: determining an operating state of the on-board medical device, sending the first prompt information in response to the environmental information exceeding the preset environmental threshold and the operating state being power on, and sending, in response to the environmental information exceeding the preset environmental threshold and the operating state being power off, the first prompt information after the operating state of the on-board medical device transitions to power on.

In an embodiment, the processor, when executing the computer program, further implements the following steps: performing registration on the first image and the second image to obtain a registered image, the registered image being an image obtained by fusing the first image and the second image in a manner of aligning feature points or target regions, and detecting quality of the registered image, to determine the check result.

In an embodiment, the processor, when executing the computer program, further implements the following steps: inputting the registered image into a pre-trained quality detection model, and detecting the inputted registered image through the quality detection model, to obtain the check result.

In an embodiment, the processor, when executing the computer program, further implements the following steps: adjusting an imaging parameter of the on-board medical device or adjusting relative positions of the first imaging assembly and the second imaging assembly.

In an embodiment, the processor, when executing the computer program, further implements the following steps: performing registration on the first image and the second image to determine a to-be-measured registration coefficient, and determining a check result according to the to-be-measured registration coefficient and a default registration coefficient.

In an embodiment, the processor, when executing the computer program, further implements the following steps: replacing the default registration coefficient with the to-be-measured registration coefficient in response to the check result indicating a failed accuracy check, or sending adjustment information to the user to instruct the user to adjust a position of the on-board medical device.

In an embodiment, the processor, when executing the computer program, further implements the following steps: determining a difference between the to-be-measured registration coefficient and the default registration coefficient, determining, in response to the difference being outside a preset difference range, that the check result indicates a failed accuracy check, determining, in response to the difference being within the preset difference range, that the check result indicates a passed accuracy check, replacing the default registration coefficient with the to-be-measured registration coefficient in response to the difference being greater than or equal to a first preset difference threshold, and sending adjustment information to the user in response to the difference being greater than or equal to a second preset difference threshold, to instruct the user to adjust a position of the on-board medical device. The second preset difference threshold is greater than the first preset difference threshold, and both the first preset difference threshold and the second preset difference threshold are outside the preset difference range.

In an embodiment, the processor, when executing the computer program, further implements the following steps: cyclically performing the step of controlling the first imaging assembly to image an imaging object to obtain a first image, and controlling the second imaging assembly to image the imaging object to obtain a second image, the step of performing an accuracy check on the on-board medical device according to the first image and the second image, to determine a check result, and the step of adjusting the on-board medical device in response to the check result indicating a failed accuracy check, until the check result indicates a passed accuracy check or a number of times the accuracy check is performed being greater than or equal to a preset number-of-times threshold, and in response to the number of times the accuracy check is performed being greater than or equal to the preset number-of-times threshold, sending second prompt information. The second prompt information is used to instruct the user to adjust the on-board medical device.

In an embodiment, the processor, when executing the computer program, further implements the following steps: extracting features of the first image and features of the second image respectively, the features of the first image being elements capable of identifying the first image, and the features of the second image being elements capable of identifying the second image, performing matching on the features of the first image and the features of the second image, and determining the to-be-measured registration coefficient according to matched features.

In an embodiment, the processor, when executing the computer program, further implements the following steps: determining a grayscale value of the first image and a grayscale value of the second image respectively, and determining the to-be-measured registration coefficient according to a similarity between the grayscale value of the first image and the grayscale value of the second image.

In an embodiment, the processor, when executing the computer program, further implements the following steps: sending third prompt information in response to the check result indicating a passed accuracy check. The third prompt information is used to prompt that environmental information of the on-board medical device acquired by the user at a current moment does not affect accuracy of the on-board medical device.

In an embodiment, the first imaging assembly is PET or SPECT, and the second imaging assembly is CT.

In an embodiment, the environmental information includes at least one of temperature information, humidity information, air pressure information, and vibration information.

In an embodiment, the environmental information includes two or more of temperature information, humidity information, air pressure information, and vibration information, and the processor, when executing the computer program, further implements the following step: sending the first prompt information in response to any one of the environmental information exceeding the preset environmental threshold.

In an embodiment, the processor, when executing the computer program, further implements the following steps:
acquiring vibration information of the on-board medical device at a current moment, and sending first prompt information in response to the vibration information exceeding a preset vibration threshold;
in response to a control operation of a user based on the first prompt information, controlling the first imaging assembly to image the imaging object to obtain the first image, and controlling the second imaging assembly to image the imaging object to obtain the second image;
performing registration on the first image and the second image to determine a to-be-measured registration coefficient; and determining a check result according to the to-be-measured registration coefficient and a default registration coefficient;
replacing the default registration coefficient with the to-be-measured registration coefficient in response to the check result indicating a failed accuracy check; or sending adjustment information to the user to instruct the user to adjust a position of the on-board medical device; and
cyclically performing the step of controlling the first imaging assembly to image an imaging object to obtain a first image, and controlling the second imaging assembly to image the imaging object to obtain a second image, the step of performing registration on the first image and the second image to determine a to-be-measured registration coefficient; and determining a check result according to the to-be-measured registration coefficient and a default registration coefficient, and the step of replacing the default registration coefficient with the to-be-measured registration coefficient in response to the check result indicating a failed accuracy check; or sending adjustment information to the user to instruct the user to adjust a position of the on-board medical device, until the check result indicates a passed accuracy check or a number of times the accuracy check is performed being greater than or equal to a preset number-of-times threshold.

The features described in the embodiments of the foregoing monitoring method for the on-board medical device are applicable to the embodiments of the computer device. For various implementations of the embodiments of the computer device, refer to the relevant description in the embodiments of the foregoing monitoring method for the on-board medical device. Details are not described herein again.

In an embodiment, a computer-readable storage medium is provided, having a computer program stored therein. When the computer program is executed by a processor, the following steps are implemented:
acquiring environmental information of the on-board medical device;
in response to the environmental information exceeding a preset environmental threshold, controlling the first imaging assembly to image an imaging object to obtain a first image, and controlling the second imaging assembly to image the imaging object to obtain a second image;
performing an accuracy check on the on-board medical device according to the first image and the second image, to determine a check result, the check result being used to evaluate a relative position relationship between the first imaging assembly and the second imaging assembly; and
adjusting the on-board medical device in response to the check result indicating a failed accuracy check.

In an embodiment, when the computer program is executed by the processor, the following steps are further implemented: sending first prompt information in response to the environmental information exceeding the preset environmental threshold, and in response to a control operation of a user based on the first prompt information, controlling the first imaging assembly to image an imaging object to obtain a first image, and controlling the second imaging assembly to image the imaging object to obtain a second image.

In an embodiment, when the computer program is executed by the processor, the following steps are further implemented: determining an operating state of the on-board medical device, sending the first prompt information in response to the environmental information exceeding the preset environmental threshold and the operating state being power on, and sending, in response to the environmental information exceeding the preset environmental threshold and the operating state being power off, the first prompt information after the operating state of the on-board medical device transitions to power on.

In an embodiment, when the computer program is executed by the processor, the following steps are further implemented: performing registration on the first image and the second image to obtain a registered image, the registered image being an image obtained by fusing the first image and the second image in a manner of aligning feature points or target regions, and detecting quality of the registered image, to determine the check result.

In an embodiment, when the computer program is executed by the processor, the following steps are further implemented: inputting the registered image into a pre-trained quality detection model, and detecting the inputted registered image through the quality detection model, to obtain the check result.

In an embodiment, when the computer program is executed by the processor, the following steps are further implemented: adjusting an imaging parameter of the on-board medical device or adjusting relative positions of the first imaging assembly and the second imaging assembly in response to the check result indicating a failed accuracy check.

In an embodiment, when the computer program is executed by the processor, the following steps are further implemented: performing registration on the first image and the second image to determine a to-be-measured registration coefficient, and determining a check result according to the to-be-measured registration coefficient and a default registration coefficient.

In an embodiment, when the computer program is executed by the processor, the following steps are further implemented: replacing the default registration coefficient with the to-be-measured registration coefficient in response to the check result indicating a failed accuracy check, or sending adjustment information to the user to instruct the user to adjust a position of the on-board medical device.

In an embodiment, when the computer program is executed by the processor, the following steps are further implemented: determining a difference between the to-be-measured registration coefficient and the default registration coefficient, determining, in response to the difference being outside a preset difference range, that the check result indicates a failed accuracy check, determining, in response to the difference being within the preset difference range, that the check result indicates a passed accuracy check, replacing the default registration coefficient with the to-be-measured registration coefficient in response to the difference being greater than or equal to a first preset difference threshold, and sending adjustment information to the user in response to the difference being greater than or equal to a second preset difference threshold, to instruct the user to adjust a position of the on-board medical device. The second preset difference threshold is greater than the first preset difference threshold, and both the first preset difference threshold and the second preset difference threshold are outside the preset difference range.

In an embodiment, when the computer program is executed by the processor, the following steps are further implemented: cyclically performing the step of controlling the first imaging assembly to image an imaging object to obtain a first image, and controlling the second imaging assembly to image the imaging object to obtain a second image, the step of performing an accuracy check on the on-board medical device according to the first image and the second image, to determine a check result, and the step of adjusting the on-board medical device in response to the check result indicating a failed accuracy check, until the check result indicates a passed accuracy check or a number of times the accuracy check is performed being greater than or equal to a preset number-of-times threshold, and in response to the number of times the accuracy check is performed being greater than or equal to the preset number-of-times threshold, sending second prompt information. The second prompt information is used to instruct the user to adjust the on-board medical device.

In an embodiment, when the computer program is executed by the processor, the following steps are further implemented: extracting features of the first image and features of the second image respectively, the features of the first image being elements capable of identifying the first image, and the features of the second image being elements capable of identifying the second image, performing matching on the features of the first image and the features of the second image, and determining the to-be-measured registration coefficient according to matched features.

In an embodiment, when the computer program is executed by the processor, the following steps are further implemented: determining a grayscale value of the first image and a grayscale value of the second image respectively, and determining the to-be-measured registration coefficient according to a similarity between the grayscale value of the first image and the grayscale value of the second image.

In an embodiment, when the computer program is executed by the processor, the following step is further implemented: sending third prompt information in response to the check result indicating a passed accuracy check. The third prompt information is used to prompt that environmental information of the on-board medical device acquired by the user at a current moment does not affect accuracy of the on-board medical device.

In an embodiment, the first imaging assembly is PET or SPECT, and the second imaging assembly is CT.

In an embodiment, the environmental information includes at least one of temperature information, humidity information, air pressure information, and vibration information.

In an embodiment, the environmental information includes two or more of temperature information, humidity information, air pressure information, and vibration information, and the processor, when executing the computer program, further implements the following step: sending the first prompt information in response to any one of the environmental information exceeding the preset environmental threshold.

In an embodiment, when the computer program is executed by the processor, the following steps are further implemented:
acquiring vibration information of the on-board medical device at a current moment, and sending first prompt information in response to the vibration information exceeding a preset vibration threshold;
in response to a control operation of a user based on the first prompt information, controlling the first imaging assembly to image the imaging object to obtain the first image, and controlling the second imaging assembly to image the imaging object to obtain the second image;
performing registration on the first image and the second image to determine a to-be-measured registration coefficient; and determining a check result according to the to-be-measured registration coefficient and a default registration coefficient;
replacing the default registration coefficient with the to-be-measured registration coefficient in response to the check result indicating a failed accuracy check; or sending adjustment information to the user to instruct the user to adjust a position of the on-board medical device; and
cyclically performing the step of controlling the first imaging assembly to image an imaging object to obtain a first image, and controlling the second imaging assembly to image the imaging object to obtain a second image, the step of performing registration on the first image and the second image to determine a to-be-measured registration coefficient; and determining a check result according to the to-be-measured registration coefficient and a default registration coefficient, and the step of replacing the default registration coefficient with the to-be-measured registration coefficient in response to the check result indicating a failed accuracy check; or sending adjustment information to the user to instruct the user to adjust a position of the on-board medical device, until the check result indicates a passed accuracy check or a number of times the accuracy check is performed being greater than or equal to a preset number-of-times threshold.

The features described in the embodiments of the foregoing monitoring method for the on-board medical device are applicable to the embodiments of the computer-readable storage medium. For various implementations of the embodiments of the computer-readable storage medium, refer to the relevant description in the embodiments of the foregoing monitoring method for the on-board medical device. Details are not described herein again.

In an embodiment, a computer program product is provided, including a computer program. When the computer program is executed by a processor, the following steps are implemented:
acquiring environmental information of the on-board medical device;
in response to the environmental information exceeding a preset environmental threshold, controlling the first imaging assembly to image an imaging object to obtain a first image, and controlling the second imaging assembly to image the imaging object to obtain a second image;
performing an accuracy check on the on-board medical device according to the first image and the second image, to determine a check result, the check result being used to evaluate a relative position relationship between the first imaging assembly and the second imaging assembly; and
adjusting the on-board medical device in response to the check result indicating a failed accuracy check.

In an embodiment, when the computer program is executed by the processor, the following steps are further implemented: sending first prompt information in response to the environmental information exceeding the preset environmental threshold, and in response to a control operation of a user based on the first prompt information, controlling the first imaging assembly to image an imaging object to obtain a first image, and controlling the second imaging assembly to image the imaging object to obtain a second image.

In an embodiment, when the computer program is executed by the processor, the following steps are further implemented: determining an operating state of the on-board medical device, sending the first prompt information in response to the environmental information exceeding the preset environmental threshold and the operating state being power on, and sending, in response to the environmental information exceeding the preset environmental threshold and the operating state being power off, the first prompt information after the operating state of the on-board medical device transitions to power on.

In an embodiment, when the computer program is executed by the processor, the following steps are further implemented: performing registration on the first image and the second image to obtain a registered image, the registered image being an image obtained by fusing the first image and the second image in a manner of aligning feature points or target regions, and detecting quality of the registered image, to determine the check result.

In an embodiment, when the computer program is executed by the processor, the following steps are further implemented: inputting the registered image into a pre-trained quality detection model, and detecting the inputted registered image through the quality detection model, to obtain the check result.

In an embodiment, when the computer program is executed by the processor, the following steps are further implemented: adjusting an imaging parameter of the on-board medical device or adjusting relative positions of the first imaging assembly and the second imaging assembly in response to the check result indicating a failed accuracy check.

In an embodiment, when the computer program is executed by the processor, the following steps are further implemented: performing registration on the first image and the second image to determine a to-be-measured registration coefficient, and determining a check result according to the to-be-measured registration coefficient and a default registration coefficient.

In an embodiment, when the computer program is executed by the processor, the following steps are further implemented: replacing the default registration coefficient with the to-be-measured registration coefficient in response to the check result indicating a failed accuracy check, or sending adjustment information to the user to instruct the user to adjust a position of the on-board medical device.

In an embodiment, when the computer program is executed by the processor, the following steps are further implemented: determining a difference between the to-be-measured registration coefficient and the default registration coefficient, determining, in response to the difference being outside a preset difference range, that the check result indicates a failed accuracy check, determining, in response to the difference being within the preset difference range, that the check result indicates a passed accuracy check, replacing the default registration coefficient with the to-be-measured registration coefficient in response to the difference being greater than or equal to a first preset difference threshold, and sending adjustment information to the user in response to the difference being greater than or equal to a second preset difference threshold, to instruct the user to adjust a position of the on-board medical device. The second preset difference threshold is greater than the first preset difference threshold, and both the first preset difference threshold and the second preset difference threshold are outside the preset difference range.

In an embodiment, when the computer program is executed by the processor, the following steps are further implemented: cyclically performing the step of controlling the first imaging assembly to image an imaging object to obtain a first image, and controlling the second imaging assembly to image the imaging object to obtain a second image, the step of performing an accuracy check on the on-board medical device according to the first image and the second image, to determine a check result, and the step of adjusting the on-board medical device in response to the check result indicating a failed accuracy check, until the check result indicates a passed accuracy check or a number of times the accuracy check is performed being greater than or equal to a preset number-of-times threshold, and in response to the number of times the accuracy check is performed being greater than or equal to the preset number-of-times threshold, sending second prompt information. The second prompt information is used to instruct the user to adjust the on-board medical device.

In an embodiment, when the computer program is executed by the processor, the following steps are further implemented: extracting features of the first image and features of the second image respectively, the features of the first image being elements capable of identifying the first image, and the features of the second image being elements capable of identifying the second image, performing matching on the features of the first image and the features of the second image, and determining the to-be-measured registration coefficient according to matched features.

In an embodiment, when the computer program is executed by the processor, the following steps are further implemented: determining a grayscale value of the first image and a grayscale value of the second image respectively, and determining the to-be-measured registration coefficient according to a similarity between the grayscale value of the first image and the grayscale value of the second image.

In an embodiment, when the computer program is executed by the processor, the following step is further implemented: sending third prompt information in response to the check result indicating a passed accuracy check. The third prompt information is used to prompt that environmental information of the on-board medical device acquired by the user at a current moment does not affect accuracy of the on-board medical device.

In an embodiment, the first imaging assembly is PET or SPECT, and the second imaging assembly is CT.

In an embodiment, the environmental information includes at least one of temperature information, humidity information, air pressure information, and vibration information.

In an embodiment, the environmental information includes two or more of temperature information, humidity information, air pressure information, and vibration information, and the processor, when executing the computer program, further implements the following step: sending the first prompt information in response to any one of the environmental information exceeding the preset environmental threshold.

In an embodiment, when the computer program is executed by the processor, the following steps are further implemented:
acquiring vibration information of the on-board medical device at a current moment, and sending first prompt information in response to the vibration information exceeding a preset vibration threshold;
in response to a control operation of a user based on the first prompt information, controlling the first imaging assembly to image the imaging object to obtain the first image, and controlling the second imaging assembly to image the imaging object to obtain the second image;
performing registration on the first image and the second image to determine a to-be-measured registration coefficient; and determining a check result according to the to-be-measured registration coefficient and a default registration coefficient;
replacing the default registration coefficient with the to-be-measured registration coefficient in response to the check result indicating a failed accuracy check; or sending adjustment information to the user to instruct the user to adjust a position of the on-board medical device; and
cyclically performing the step of controlling the first imaging assembly to image an imaging object to obtain a first image, and controlling the second imaging assembly to image the imaging object to obtain a second image, the step of performing registration on the first image and the second image to determine a to-be-measured registration coefficient; and determining a check result according to the to-be-measured registration coefficient and a default registration coefficient, and the step of replacing the default registration coefficient with the to-be-measured registration coefficient in response to the check result indicating a failed accuracy check; or sending adjustment information to the user to instruct the user to adjust a position of the on-board medical device, until the check result indicates a passed accuracy check or a number of times the accuracy check is performed being greater than or equal to a preset number-of-times threshold.

The features described in the embodiments of the foregoing monitoring method for the on-board medical device are applicable to the embodiments of the computer program product. For various implementations of the embodiments of the computer program product, refer to the relevant description in the embodiments of the foregoing monitoring method for the on-board medical device. Details are not described herein again.

Those of ordinary skill in the art may understand that some or all procedures in the methods in the foregoing embodiments may be implemented by a computer program instructing related hardware, the computer program may be stored in a non-transitory computer-readable storage medium, and when the computer program is executed, the procedures in the foregoing method embodiments may be implemented. Any reference to the memory, database, or other media used in the embodiments provided in the present disclosure may include at least one of a non-transitory memory and a transitory memory. The non-transitory memory may include a read-only memory (ROM), a magnetic tape, a floppy disk, a flash memory, an optical memory, a high-density embedded non-transitory memory, a resistive random access memory (ReRAM), a magnetoresistive random access memory (MRAM), a ferroelectric random access memory (FRAM), a phase change memory (PCM), a graphene memory, and the like. The transitory memory may include a random access memory (RAM), an external cache memory, or the like. As an illustration but not a limitation, the RAM may be in a variety of forms, such as a static random access memory (SRAM) or a dynamic random access memory (DRAM). The database as referred to in the embodiments provided in the present disclosure may include at least one of a relational database and a non-relational database. The non-relational database may include a blockchain-based distributed database or the like, and is not limited thereto. The processor as referred to in the embodiments provided in the present disclosure may be a general-purpose processor, a central processing unit, a graphics processor, a digital signal processor, a programmable logic device, a quantum computing-based data processing logic device, or the like, and is not limited thereto.

Technical features in the foregoing embodiments may be randomly combined. To make the description concise, not all possible combinations of the technical features in the foregoing embodiments are described. However, the combinations of these technical features shall be considered as falling within the scope recorded by this specification provided that no conflict exists.

The foregoing embodiments merely express several implementations of the present disclosure. The description thereof is relatively specific and detailed, but cannot be understood as limitations on the patent scope of the present disclosure. It should be noted that for those of ordinary skill in the art, several transforms and improvements can be made without departing from the concept of the present disclosure, all of which fall within the protection scope of the present disclosure. Therefore, the protection scope of the present disclosure shall be subject to the appended claims.

What is claimed is:

1. A monitoring method for an on-board medical device, wherein the on-board medical device comprises at least a first imaging assembly and a second imaging assembly, and the method comprises:
acquiring environmental information of the on-board medical device;
in response to the environmental information exceeding a preset environmental threshold, controlling the first imaging assembly to image an imaging object to obtain a first image, and controlling the second imaging assembly to image the imaging object to obtain a second image;
performing an accuracy check on the on-board medical device according to the first image and the second image, to determine a check result, the check result being used to evaluate a relative position relationship between the first imaging assembly and the second imaging assembly; and
adjusting the on-board medical device in response to the check result indicating a failed accuracy check.

2. The method according to claim 1, wherein in response to the environmental information exceeding the preset environmental threshold, controlling the first imaging assembly to image the imaging object to obtain the first image, and controlling the second imaging assembly to image the imaging object to obtain the second image comprises:
sending first prompt information in response to the environmental information exceeding the preset environmental threshold; and
in response to a control operation of a user based on the first prompt information, controlling the first imaging assembly to image the imaging object to obtain the first image, and controlling the second imaging assembly to image the imaging object to obtain the second image.

3. The method according to claim 1, wherein performing the accuracy check on the on-board medical device according to the first image and the second image, to determine the check result comprises:
performing registration on the first image and the second image to obtain a registered image, the registered image being an image obtained by fusing the first image and the second image in a manner of aligning feature points or target regions; and
detecting quality of the registered image, to determine the check result.

4. The method according to claim 3, wherein detecting the quality of the registered image comprises:
inputting the registered image into a pre-trained quality detection model, and detecting the inputted registered image through the quality detection model, to obtain the check result.

5. The method according to claim 1, wherein the adjusting the on-board medical device in response to the check result indicating the failed accuracy check comprises:
in response to the check result indicating the failed accuracy check:
adjusting an imaging parameter of the on-board medical device, or
adjusting relative positions of the first imaging assembly and the second imaging assembly.

6. The method according to claim 1, wherein performing the accuracy check on the on-board medical device according to the first image and the second image, to determine the check result comprises:
performing registration on the first image and the second image to determine a to-be-measured registration coefficient; and
determining the check result according to the to-be-measured registration coefficient and a default registration coefficient.

7. The method according to claim 6, wherein adjusting the on-board medical device in response to the check result indicating the failed accuracy check comprises:
in response to the check result indicating the failed accuracy check, replacing the default registration coefficient with the to-be-measured registration coefficient, or sending adjustment information to the user to instruct the user to adjust relative positions of the first imaging assembly and the second imaging assembly.

8. The method according to claim 6, wherein determining the check result according to the to-be-measured registration coefficient and the default registration coefficient comprises:
determining a difference between the to-be-measured registration coefficient and the default registration coefficient;
determining, in response to the difference being outside a preset difference range, that the check result indicates the failed accuracy check; and
determining, in response to the difference being within the preset difference range, that the check result indicates a passed accuracy check.

9. The method according to claim 8, wherein adjusting the on-board medical device in response to the check result indicating the failed accuracy check comprises:
replacing the default registration coefficient with the to-be-measured registration coefficient in response to the difference being greater than or equal to a first preset difference threshold; and
sending adjustment information to the user in response to the difference being greater than or equal to a second preset difference threshold, to instruct the user to adjust a position of the on-board medical device; the second preset difference threshold being greater than the first preset difference threshold, and both the first preset difference threshold and the second preset difference threshold being outside the preset difference range.

10. The method according to claim 1, wherein the method further comprises:
cyclically performing the step of controlling the first imaging assembly to image an imaging object to obtain a first image, and controlling the second imaging assembly to image the imaging object to obtain a second image, the step of performing an accuracy check on the on-board medical device according to the first image and the second image, to determine a check result, and the step of adjusting the on-board medical device in response to the check result indicating a failed accuracy check, until the check result indicates a passed accuracy check or a number of times the accuracy check is performed being greater than or equal to a preset number-of-times threshold, until the check result indicates a passed accuracy check or a number of times the accuracy check is performed being greater than or equal to the preset number-of-times threshold.

11. The method according to claim 10, wherein the method further comprises:
in response to the number of times the accuracy check is performed being greater than or equal to the preset number-of-times threshold, sending second prompt information, the second prompt information being used to instruct the user to adjust the on-board medical device.

12. The method according to claim 2, wherein sending the first prompt information in response to the environmental information exceeding the preset environmental threshold comprises:
determining an operating state of the on-board medical device;
sending the first prompt information in response to the environmental information exceeding the preset environmental threshold and the operating state being power on; and
sending, in response to the environmental information exceeding the preset environmental threshold and the operating state being power off, the first prompt information after the operating state of the on-board medical device transitions to power on.

13. The method according to claim 6, wherein performing registration on the first image and the second image to determine the to-be-measured registration coefficient comprises:
extracting features of the first image and features of the second image respectively, the features of the first image being elements capable of identifying the first image, and the features of the second image being elements capable of identifying the second image;
performing matching on the features of the first image and the features of the second image; and
determining the to-be-measured registration coefficient according to matched features.

14. The method according to claim 6, wherein performing registration on the first image and the second image to determine the to-be-measured registration coefficient comprises:
determining a grayscale value of the first image and a grayscale value of the second image respectively; and
determining the to-be-measured registration coefficient according to a similarity between the grayscale value of the first image and the grayscale value of the second image.

15. The method according to claim 1, further comprising:
sending third prompt information in response to the check result indicating a passed accuracy check, the third prompt information being used to prompt that environmental information of the on-board medical device acquired by a user at a current moment does not affect accuracy of the on-board medical device.

16. The method according to claim 1, wherein the first imaging assembly is positron emission tomography (PET) or single-photon emission computed tomography (SPECT), and the second imaging assembly is computed tomography (CT).

17. The method according to claim 1, wherein the environmental information comprises at least one of temperature information, humidity information, air pressure information, and vibration information.

18. The method according to claim 1, wherein the environmental information comprises two or more of temperature information, humidity information, air pressure information, and vibration information; and
sending first prompt information in response to the environmental information exceeding the preset environmental threshold, comprising:
sending the first prompt information in response to any one of the environmental information exceeding the preset environmental threshold.

19. An on-board medical system, comprising an on-board medical device, a control device, and an environmental detection device that are communicatively connected with one another; wherein the environmental detection device is configured to detect environmental information of a mobile carrier where the on-board medical device is located, and send the environmental information to the control device; and
the control device comprises a memory and a processor, the memory storing a computer program, wherein the processor, when executing the computer program, implements steps of the method according to claim 1.

20. A computer device, comprising a memory and a processor, the memory storing a computer program, wherein the processor, when executing the computer program, implements steps of the method according to claim 1.
